(19) United States
(12) Patent Application Publication
(10) Pub. No.: US 2003/0106556 A1
(43) Pub. Date: Jun. 12, 2003

(54) Respiratory nasal filter

(76) Inventors: Vladimir Alperovich, Modiin (IL); Valery Peary, Ramat-Gan (IL); Michael Gershman, Zoran (IL); Simon Litvin, Newton, MA (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/148,973

(22) PCT Filed: Dec. 8, 2000
(86) PCT No.: PCT/US00/42691

(30) Foreign Application Priority Data
Dec. 10, 1999 (RU) 99126892

(51) Int. Cl. A61M 16/00

(52) U.S. Cl. 128/206.11; 128/205.27

(57) ABSTRACT

The device claimed refers to means for human respiration organ protection against dust-like particles—in particular, against allergen-carrying particles. It may be used as a nasal filter, as a filter in respirators, and so forth. The device comprises a hollow body having one or several inlet channels and one outlet orifice that are intended for the passage of inhaled air, with the inner surface of said body having such a shape that is close to the shape of a truncated cone, and said inlet channels are made in the major base of the cone with the direction of the axis of each of the channels being combined out of the tangential component and axial component, while the outlet orifice is made in the vertex (minor base) of the cone, and the inner body surface is covered with a sticky substance capable of retaining dust and allergen particles contained in the inhaled air. Cleaning of inhaled air takes place in filter's “vortex chamber” formed by the body of the above-indicated shape. When air enters this cone, it swirls on a spiral path owing to the configuration and direction of inlet channels. When dust particles get into spirally moving airflows, they shift to filter body walls because of the action of centrifugal forces, encounter said walls and get deposited on sticky substance that covers the entire filter body from the inside. The filter is designed so that no noticeable resistance to inhaling and exhaling is produced.

### FIELD OF THE INVENTION

The invention applied herein refers to the field of human respiratory system protection and enables one to perform effective purification of inhaled air by removing dust-like particles from inhaled air (in particular, allergen-carrying particles) without any noticeable additional resistance to inhalation and exhalation. The invention can be used as a nasal filter, in respirators and so forth.

### BACKGROUND OF THE INVENTION

A respiratory filter is known [WO 86/04249] made in the form of two nasal capsules containing filtering elements impregnated with deodorants or medicinal preparations. Capsules should be inserted into nostrils. Air purification is carried out owing to the fact that inhaled air passes through said filtering elements.

Another respiratory filter is known—“Intra-nasal filter” [U.S. Pat. No. 5,117,820], shaped an elongated cylinder made out of synthetic spongy material. After being compressed radially this filter takes the shape of a cylinder of such a diameter that it can be installed in human nasal cavity. When inside human nasal cavity, the filter material expands and fills the nasal cavity space. Air purification in this filter is attained owing to the fact that the air inhaled passes through filtering elements out of synthetic spongy material, in which the particles get captured.

Another respiratory filter is known—“Nasal filter” [U.S. Pat. No. 3,747,597]. It has filters to be inserted in nasal cavities (nostrils). The inner cavity of each filter is of a shape close to truncated cone shape. A spherical filtering element is placed inside filter inner cavity. This filter operates in the same manner as a conical valve does—i.e. in the process of exhalation the spherical filtering element that is located inside filter cavity shifts to the major cone base, which position doesn't impede the passage of exhaled air, while in the process of inhalation said spherical filtering element shifts to the minor cone base thus “plugging” the orifice in this base, owing to which fact the inhaled air passes only through said spherical filtering element.

Another respiratory filter is known—[GB App. No 2216806] made in the form of a device comprising inserts (to be inserted in nostrils) and having hollow tubular bodies with an internal flange for holding filtering element in place. When joined together the inserts form a clamp that serves for fixing filter to nasal septum. Air cleaning is performed due to the fact that in the course of inhalation the air passes through filtering elements consisting of hollow bodies with inserts in them.

Air cleaning in the above-mentioned filters is attained due to the fact that in the course of inhalation the air passes through filtering elements made out of different materials leaving atmospheric contaminants and allergens on said elements. Filtering elements represent the main component of the above-mentioned filters. However, the use of said filtering elements impedes breathing. This is so because filtering elements impede the free access of air flow producing considerable resistance to the air flow inhaled.

The closest analogue (in terms of engineering essence) to the invention being claimed is a respiratory filter “Nasal and oral filters” [U.S. Pat. No. 5,787,884], which is characterized by the use of air-passage channel free from filtering materials. This filter is designed for the purpose of protecting respiratory airways against dust and allergens contained in the inhaled air. The filter is shaped so that it is possible to accommodate it in human mouth or nasal cavity (in the latter case it tightly adjoins the inner surface of nasal cavity due to the elasticity of its body). An air-passage channel is arranged inside the filter in such a manner that it enables human beings to breathe easily. This channel is of curvilinear shape and has special dust particulate entrapment zones located in the immediate vicinity of places where said air-passage channel changes its direction. Owing to the curvilinear shape of the air-passage channel, the inhaled air changes the direction of its motion several times. Dust particles contained in the inhaled air get into entrapment zones where they get retained due to the fact that these zones are covered with a sticky substance.

The motion of airflow is not that impeded in this design as compared to designs with filtering inserts. However, the air-passage channels in this filter are made narrow and tortuous for the purpose of increasing the probability of the entrapment of dust particles, which fact produces noticeable resistance to airflow passing through the filter (though this resistance is much lower than the one produced in the case when filters with inserts are used). Besides, as the filter gets more and more clogged in the course of its operation, the resistance to airflow motion increases.

### SUMMARY OF THE INVENTION

The problem to be solved with the help of the invention claimed herein consists in creating such a respiratory filter that could be used as a respiratory protective device (that protects human respiratory organs against dust particles—in particular, allergen-carrying particles) that is characterized by low aerodynamic resistance and that is inconspicuous (when being used) for other people.

The essence of the present invention consists in the fact that in a respiratory filter comprising a body that has one or several inlet channels and one outlet orifice that are intended for the passage of the inhaled air, in accordance with the invention the body is made hollow with inner surface of said body having such a shape that is close to truncated cone shape, and said inlet channels are made in the major cone base with the direction of the axis of each channel being combined out of the tangential and axial components, while the outlet orifice is made in the vertex (minor base) of the cone, and the inner body surface is covered with a sticky substance capable of retaining dust and allergen particles contained in the inhaled air.

Cleaning of the inhaled air in the device claimed herein takes place in filter's “vortex chamber” formed by the body (the shape of which is close to that of a truncated cone). When air enters this cone it swirls on a spiral path owing to the configuration of inlet channels in the area of major cone base. The channels are arranged so that the direction of their axes is combined out of two components—namely, axial component and tangential component. When dust particles get into spiral-like streams they shift to body walls due to the action of centrifugal forces, encounter said body walls and stay on the sticky substance that covers the entire body from the inside.

For the sake of convenience in using the device claimed as a nasal filter its body may have a shape bent in longitudinal direction and/or oval in cross section—in other words, a shape that is close to the one of nasal cavity.

To provide for the comfortable accommodation of the filter in the nasal cavity, its body may be made of elastic material. Siloxane elastomer (for example, siloxane rubber) widely used in articles for medical purposes could be chosen as such material [Chemical Encyclopedia. —Moscow: Sovetskaya Encyclopedia Publishing-House, 1990, vol.2, p. 510].

In addition, the body may be made out of porous hydrophilic material (for instance, polyurethane foam). Absorbing the surplus moisture present in the nasal cavity such material would enhance the convenience in using the filter.

In order to provide for the reliable accommodation of the filter in the nasal cavity and for the reliable sealing of the gap between filter body and inner surface of nasal cavity, the outer surface of the body has a special sealing collar made out of elastic or ductile material. This material could be also porous and hydrophilic—to absorb the surplus of moisture produced in the nasal cavity. Siloxane elastomer could be used as an elastic material, while silicone-acryl latex could be used as a ductile material [Chemical Encyclopedia. —Moscow: Sovetskaya Encyclopedia Publishing-House, 1990, vol.2, p. 511].

In addition, the major cone base may have a convex shape on the outer side, while the inlet channels may be made so that they partially pass through the side part of the body. Such an arrangement would provide for the greater area of air intake and for smoother flowing of the air stream into the filter (in other words, without sharp turn of speed vector). In the final end such a geometry of air intake makes it possible to reduce resistance to breathing by 10-15%.

In order to decrease the number of particles that haven't been entrapped by the sticky layer on the inner surface of the body, a special collar is made around the outlet orifice, said collar being directed into the inside of the body. This collar, together with vertex (minor base) of the cone and inner wall of the body, forms the inner ring-shaped chamber. The particles that haven't deposited on body walls get entrapped in this chamber.

Also, for the purpose of better retention of dust particles the surface of the inner ring-shaped chamber can be covered with said sticky substance.

The surface of air inlet channels as well as the outer surface of the major base of the cone-like body can be also covered with said sticky substance for the same purpose.

Electrostatic ointment can be used for example as said sticky substance that is applied onto the inner surface of the body, ring-shaped chamber surface, and air inlet channels [Electrets. /Under the editorship of G. Sessler Translated from English. —Moscow, 1983]. Such ointment can be made based on polymers, salts, and co-polymers having hydrophilic and hydrophobic bonds. Glycerin that is characterized by stickiness, that doesn't dry out and that performs well in wetting dust can be used as the basis for a sticky substance composition.

Filter body can be made out of a material having permanent electrostatic charge—i.e. electret [Electrets /Under the editorship of G. Sessler. Translated from English. —Moscow, 1983]. In this case electrostatic charge of the body promotes the polarization of dust particles and facilitates the attraction of said particles to filter body.

One or several protrusions are made on the inner surface of the body (somewhere in the halfway of filter length) for the purpose of separating small particles, the most part of which stay in the axial airflow due to their low weight. These protrusions are oriented transverse to the spiral-like lines that follow the directions of the axes of air inlet channels over the inner surface of the body. These protrusions have the shape of a convex part of an aerodynamic wing in the cross section. Geometric size of said protrusions is chosen based on the following relations:

0.1R<h<0.3R;

0.25H<L<0.8H,

where:

h—protrusion height in the radial direction;

R—maximum radial size of the inner space of the body;

L—projection of the protrusion onto the body axis;

H—length of the inner space of the body.

When the air stream flows around the protrusion, a low pressure area is produced immediately above the protrusion (see, for example, [L. G. Loytsyanskiy. Fluid and Gas Mechanics. —Moscow, Nauka, 1970, p.211]. Owing to this area, air streams with small particles get shifted from the axis to body walls where said small particles get entrapped by the sticky layer.

Besides, said protrusions retard the circular motion of air stream in the area of filter outlet orifice without producing additional turbulence. Reduction in aerodynamic resistance of the filter (and, hence, reduction in resistance to breathing) is attained due to the retardation of the vortex flow.

The above-indicated geometric relations for protrusion parameters were obtained experimentally. When the protrusion height in the radial direction (h) is greater than 0.3R, the turbulence of the airflow increases sharply in the entire space of the filter, thus adversely affecting the cleaning of inhaled air. When h is less than 0.1R, the above-indicated effect of reduction in aerodynamic resistance of the filter is not observed. When L>0.8H (that is, when the protrusion extends along almost the entire body), the protrusion actually partitions a certain area off where air stagnates and dust gets accumulated. This would go on until the discharge of this extremely dusty air occurs, and this dusty air will be picked up by the main stream and then carried into the filter outlet orifice. Besides, when L>0.8H, the protrusion begins to impede the normal swirling of the airflow. When the value of L is less than 0.25H, the above-indicated effect of the reduction in aerodynamic resistance of the filter is not observed.

In terms of manufacture technology said protrusions can be made by the way of residual deformation of the body.

For the sake of convenience in using the device claimed herein as a nasal filter, it may consist of two bodies connected by a flexible bridge. In this case better attachment of the filter and mutual orientation of bodies in nasal cavities are provided for. For the purpose of better adjoining to the outer nose surface and greater degree of inconspicuousness said bridge may be made in the form of a transparent film with a an adhesive layer.

### BRIEF DESCRIPTION OF THE DRAWINGS

The essence of the present invention is illustrated by the following figures:

FIG. 1—appearance of the filter claimed herein;

FIG. 2—sectional view of the filter claimed herein;

FIG. 3—an example showing the placement of the filter in the nasal cavity;

FIG. 4—two filters joined together by a flexible bridge.

### DETAILED DESCRIPTION OF PREFERRED EMBODIMENTS

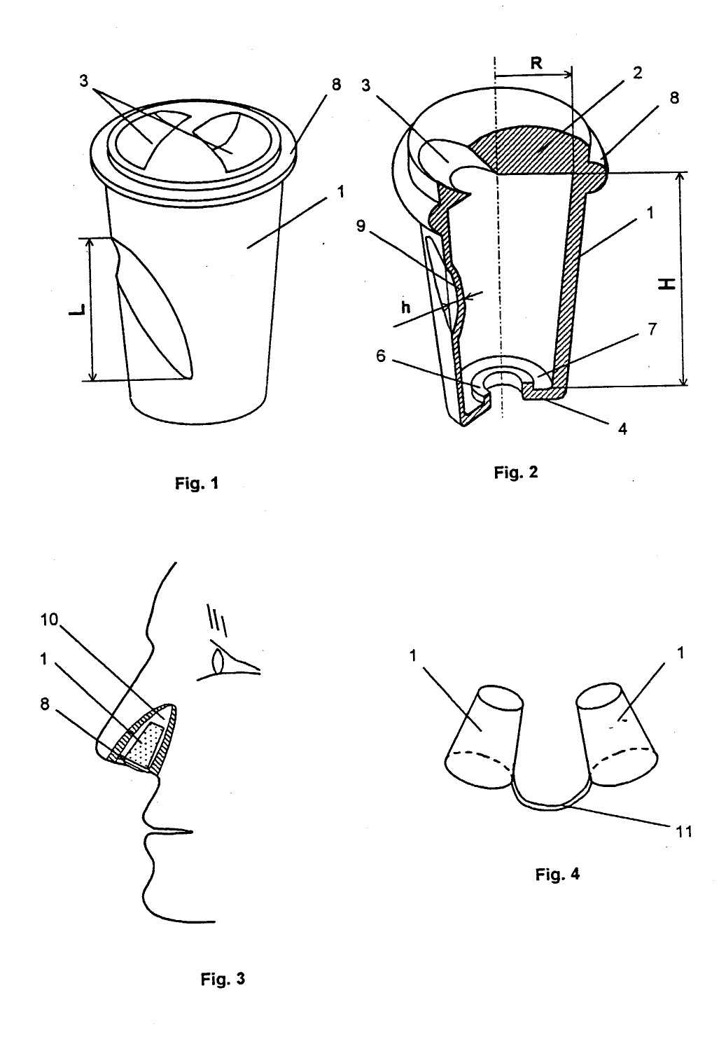

The filter claimed herein (FIG. 1, and FIG. 2) comprises a body 1 having a shape of truncated cone. Air inlet channels 3 (intended for the passage of the inhaled air) are made in the major cone base 2 of body 1 (said major cone base is of convex shape from the outside). The direction of the axis of each channel 3 is combined out of tangential component and axial components, thus providing for the passage of inhaled air in the proper direction. Vertex (minor base) of cone 4 of body 1 has an outlet orifice 5, around which collar 6 is made in such a way that said collar together with inner surface of body 1 forms inner ring-shaped chamber 7. Sealing ring 8 is located on the outer surface of body 1 (closer to major cone base 2), while protrusion 9 is made on the inner surface of body 1. In the cross section, said protrusion has the shape of the convex part of aerodynamic wing. Geometric sizes of protrusion 9 are chosen based on relationships indicated above.

Inner surfaces of body 1, inlet channels 3 and inner ring-shaped chamber 7 are covered with a sticky layer—in particular, with polyisobutylene.

FIG. 3 illustrates the placement of the filter claimed herein in nasal cavity 10. Sealing ring 8 serves provides for the gasketing and fixing of the filter.

FIG. 4 shows two bodies 1 of nasal filters connected with a flexible bridge 11.

In the course of inhalation the air with dust particles is drawn in the filter through inlet channels 3 made in major cone base 2 of body 1 and gets into the inner space of body 1. In this process the inhaled air gets swirled and moves inside body 1 on a spiral-like path due to the indicated configuration of channels 3. Under the action of centrifugal force that emerge when air moves in such a manner, dust particles shift to the periphery of the flow, encounter the walls of body 1 and get entrapped by sticky layer covering said walls. Deposition of dust particles onto sticky layer takes place over the entire length of filter body 1. Dust particles that are not entrapped by sticky walls of body 1 get into the inner ring-shaped chamber 7 also covered with sticky layer.

Due to the turbulence of the airflow in the axial area of body 1, a portion of small-sized dust may be carried away by the airflow through orifice 5 to the nasal cavity. One or several protrusions 9 made on the inner surface of body 1 and oriented transversely to the direction of the airflow serve to prevent this from happening. When the airflow encounters protrusion 9, a low-pressure area is produced above protrusion 9, and said low-pressure area draws the turbulent airflow together with small-sized dust in to the periphery. In parallel with this, protrusions 9 retard the circular motion of the airflow in the area of the vertex (minor base) of cone 4 with no additional turbulence being produced. Approximately 1.5-fold reduction in aerodynamic resistance of the filter (and, hence, reduction in resistance to breathing) is attained due to the effect of the retardation of the vortex flow.

Testing of the filter was conducted at a specially designed test bed. Wheat flour (of fineness 10÷30 μm) and street dust (of fineness 3÷15 μm) mixed with pollen (of fineness 15÷25 μm) as well as cacao powder (of fineness 5÷20 μm) were used for the purpose of simulating dust. Dust was placed into a container, out of which it was then ejected by a stream of air into the separator. The separator served to divide air+dust mixture into two equal flows. The equality of flows (i.e. the equality of volumes per unit of time) was monitored with the aid of flowmeters (the flowmeters manufactured by Manoraz Ltd, Israel were used in the course of testing). One of the flows was then directed to the first paper filter, while the second flow was directed through the nasal filter being tested to the second paper filter. The motion of both airflows was effected due to the rarefaction produced by an exhaust fan.

The efficiency of dust entrapment (E) was determined by two methods:

1) by weighing the dust that was deposited on the first and second filters; in this case the efficiency of dust entrapment was determined by the following formula:

E=100(1−W₁/W₂)[%],

where

W₁ and W₂—weight of dust that was deposited on the first and second filter. respectively;

2) by visual count of the number of particles that were deposited on the first and second filters (these particles were observed with the aid of a microscope and digital photo camera connected to a computer). In this case the efficiency of dust entrapment was determined by the following formula:

E=100(1−N₁/N₂)[%],

where

N₁ and N₂—number of particles that were deposited on the first and second filter, respectively.

Using both above-described methods we have established that the nasal filter tested enables one to remove particles of size 10 μm and over from the air (i.e. to clean air of particles of this size) with an efficiency of 88÷100%. The accuracy of measurement was 1%.

Extra resistance produced by the filter in a nose was measured as the difference in pressure drop in a nose mock-up with a filter and the one without a filter. As a result of these measurements, we have found out that with a flow of 15 liters per minute for two nostrils the resistance of the filter doesn't exceed 5 mm of water column (50 Pa), which corresponds to sanitary requirements and standards for respiratory devices.

The tested version of nasal filter has the shape of a truncated cone with the following dimensions:

Major base of the cone—an oval 9×10 mm in size;

Vertex (minor base) of the cone—an oval 6×9 mm in size;

Height—13 mm.

The filter body had two spiral protrusions of the following size:

length of the projection of the protrusion on filter axis (L)—4 mm;

protrusion width—3 mm:

protrusion height (in the radial direction)—(h)—1 mm.

Having the above-indicated geometric dimensions, the filter was capable of catching up to 4 mg of dust with no deterioration of air cleaning parameters and resistance to the airflow. With mean dust content in the air being in the range 1÷2 mg/m³ one filter can be effectively used for over 8 hours. At the same time the filter made, for example, out of thermosoftening plastic by injection casting method is so inexpensive that the frequency of filter replacement will be mainly determined by hygienic considerations and convenience for uses rather than by filter capacity in terms of entrapped dust.

Thus, the filter claimed herein is an effective and efficient device for protecting human respiratory airways against dust particles contained in the air (for instance, against pollen), the size of which lies within the range 10÷50 μm.

The filter claimed herein is convenient in use and meets aesthetic requirements. This filter doesn't cause inconvenience to human beings that use it, because the air-passage channel is free from the filtering inserts. Hermetic sealing and no irritation in the nasal cavity are ensured due to the fact that the filter has soft elastic or ductile sealing layer or by the fact that the entire filter could be made out of elastic material. Filter size and dark color make it practically inconspicuous for other people.

What is claimed is:

1. A respiratory filter comprising a hollow body having one or several inlet channels and one outlet orifice that are intended for the passage of inhaled air, wherein the inner surface of said body having such a shape that is close to the shape of a truncated cone, and said inlet channels are made in the major base of the cone with the direction of the axis of each of the channels being combined out of the tangential component and axial component, while the outlet orifice is made in the vertex (minor base) of the cone, and the inner body surface is covered with a sticky substance capable of retaining dust and allergen particles contained in the inhaled air.

2. The respiratory filter of claim 1, wherein the body has a shape bent in the longitudinal direction and/or a shape oval in the cross section close in configuration to the shape of nasal cavity.

3. The respiratory filter of claim 1, wherein the body is made out of elastic material.

4. The respiratory filter of claim 3, wherein the body is made out of porous hydrophilic material.

5. The respiratory filter of claim 1, wherein the outer surface of the body has a sealing ring made out of elastic or ductile material.

6. The respiratory filter of claim 5, wherein the sealing ring is made out of porous hydrophilic material.

7. The respiratory filter of claim 1, wherein the major base of the cone is of convex shape on the outside of the body.

8. The respiratory filter of claim 1, wherein the inlet channels may be made so that they partially pass through the side part of the body.

9. The respiratory filter of claim 1, wherein an electrostatic ointment is used as the said sticky substance.

10. The respiratory filter of claim 1, wherein a special collar directed into the inside of the filter body is made around the outlet orifice, said collar, together with the vertex (minor base) of the cone and inner wall of the body, forms an inner ring-shaped chamber.

11. The respiratory filter of claim 10, wherein the surface of the inner ring-shaped chamber is covered with said sticky substance.

12. The respiratory filter of claim 11, wherein an electrostatic ointment is used as said sticky substance.

13. The respiratory filter of claim 1, wherein the surface of inlet channels and/or the outer surface of major base of the cone-shaped body base are covered with said sticky substance.

14. The respiratory filter of claim 13, wherein an electrostatic ointment is used as the said sticky substance.

15. The respiratory filter of claim 1, wherein said filter is made out of a material having permanent electrostatic charge (for example, electret).

16. The respiratory filter of claim 1, wherein one or several protrusions are made on the inner surface of the filter body (somewhere in the halfway of filter length), and said protrusions are oriented transverse to the spiral lines that follow the directions of the axes of air inlet channels over the inner surface of the body, with said protrusions having the shape of a convex part of an aerodynamic wing in the cross section, with geometric size of said protrusion(s) being chosen based on the following relations:
0.1R<h<0.3R;
0.25H<L<0.8H,
where:
h—protrusion height in the radial direction;
R—maximum radial size of the inner space of the filter body;
L—projection of the protrusion onto the filter body axis,
H—length of the inner space of the filter body.

17. The respiratory filter of claim 16, wherein said protrusion is produced by the way of residual deformation of filter body.
